(19) United States
(12) Patent Application Publication
(10) Pub. No.: US 2011/0137184 A1
(43) Pub. Date: Jun. 9, 2011

(54) PRESSURE SENSING

(76) Inventors: Fan Ren, Gainesville, FL (US); Stephen John Pearton, Gainesville, FL (US)

(21) Appl. No.: 13/058,700

(22) PCT Filed: Aug. 18, 2009
(86) PCT No.: PCT/US09/54139

§ 371 Date: Feb. 11, 2011

Related U.S. Application Data
(60) Provisional application No. 61/090,154, filed on Aug. 19, 2008.

(51) Int. Cl. A61B 5/021; H01L 29/778; H01L 29/205

(52) U.S. Cl. 600/485; 257/194; 257/76; 257/E29.246; 257/E29.091

(57) ABSTRACT

A high electron mobility transistor (HEMT) is disclosed capable of performing as a pressure sensor. In one embodiment, the subject pressure sensor can be used for the detection of body fluid pressure. A piezoelectric, biocompatible film can be used to provide a pressure sensing functionalized gate surface for the HEMT. Embodiments of the disclosed sensor can be integrated with a wireless transmitter for constant pressure monitoring.

### CROSS-REFERENCE TO RELATED APPLICATION

The present application claims the benefit of U.S. Provisional Application Ser. No. 61/090,154, filed Aug. 19, 2008, which is hereby incorporated by reference in its entirety, including any figures, tables, or drawings.

### BACKGROUND OF INVENTION

Often, the blood pressure of patients in the hospital needs to be constantly monitored. Blood pressure can be measured invasively or non-invasively. The typical invasive technology involves a cannula needle placed in an artery, a fluid flow system receiving blood from the cannula, and an electronic transducer reading the blood pressure. The current non-invasive technology involves a pressure cuff placed on the patient's arm, an air pump providing air to the pressure cuff, and a signal line reading the blood pressure.

### BRIEF SUMMARY

Embodiments of the present invention relate to a high electron mobility transistor (HEMT) capable of performing as a pressure sensor. In a specific embodiment, the HEMT can be used for the detection of body fluid pressure.

According to an embodiment of the invention, a piezoelectric, biocompatible film can be provided on a gate region of the HEMT. In a specific embodiment, the piezoelectric, biocompatible film can be polyvinylidene fluoride (PVDF). In another specific embodiment, the piezoelectric, biocompatible film can be one or more metal oxides with piezoelectric properties.

Embodiments of the disclosed sensor can be integrated with a wireless transmitter for constant pressure monitoring and reporting. Embodiments incorporating a wireless transmitter can reduce or eliminate lines connected to a patient, reducing emotional turmoil and reducing the possibility of mechanical problems.

In one embodiment, the subject pressure sensor can be mounted on the head of intravenous therapy or IV therapy for continual measurement of the pressure of the blood vessel. Further implementations of the subject device can also be used to monitor other body fluids, such as cerebrospinal fluid in the brain.

### BRIEF DESCRIPTION OF DRAWINGS

FIGS. 1A-1F show a cross-sectional view of an AlGaN/GaN HEMT sensor for illustrating a fabricating process according to an embodiment of the invention.

FIGS. 2A-2B are optical microscope images; FIG. 2A shows a top view of a gate region without PVDF; and FIG. 2B shows a top view of a gate region with PVDF according to an embodiment of the present invention.

FIG. 3 shows a plot of time dependent drain current of a PVDF gated AlGaN/GaN HEMT of an embodiment of the present invention exposed to different pressures.

FIG. 4 shows a plot of drain current change of a PVDF gated AlGaN/GaN HEMT of an embodiment of the present invention as a function of pressure.

FIG. 5 shows a plot of the drain current of a PVDF gated AlGaN/GaN HEMT of an embodiment as a function of pressure. The PVDF was polarized by grounding a copper chuck holding the sample and applying 10 kV to the copper wire electrode.

FIG. 6 shows a plot of the drain current of a PVDF gated AlGaN/GaN HEMT of an embodiment as a function of pressure. The PVDF was polarized by grounding the copper wire electrode and applying 10 kV to the copper chuck holding the sample.

FIG. 7 shows a plot of the drain current of a PVDF gated AlGaN/GaN HEMT of an embodiment as a function of chamber pressure.

### DETAILED DISCLOSURE

Embodiments of the present invention relate to a high electron mobility transistor (HEMT) capable of performing as a pressure sensor. Certain embodiments can be used for the detection of body fluid pressure. In an embodiment of the invention, the subject sensor can be utilized in a blood pressure detector. In one embodiment, the sensor can be portable. In another embodiment, the sensor can be implantable.

According to an embodiment of the invention, a piezoelectric, biocompatible film can be provided on a gate region of the HEMT. The gate region of the HEMT can be a sensing region. In a specific embodiment, the piezoelectric, biocompatible film can be polyvinylidene fluoride (PVDF). In another embodiment, the piezoelectric, biocompatible film can be a metal oxide having piezoelectric properties.

Transducer materials for converting one form of energy into another form of energy can be used in a variety of sensing applications. The conversion of pressure into electric potential is referred to as a piezoelectric effect. Many materials in nature exhibit a weak piezoelectric effect under appropriate conditions, including whale bone and tendon. Some oxide materials possess piezoelectric properties that can also be used in different types of pressure sensors. In addition, there are organic molecules with polar bonds which exhibit piezoelectricity. Polyvinylidene fluoride (PVDF) film is a piezoelectric and biocompatible film that can be used in pressure sensors. Polarized PVDF is flexible, has low density and low mechanical impedance, and is easy to fabricate as a ferroelectric. The size of a typical PVDF sensor is in the range of a few centimeters square or larger. By integrating PVDF with a high electron mobility transistor (HEMT), embodiments of the present invention can provide miniaturization of a pressure detection device and high sensitivity to pressure for many applications, including medical applications.

According to embodiments of the present invention, variations in ambient pressure can induce changes in the charge in the polarized PVDF, leading to a change of surface charge in the gate region of the HEMT. This gate potential change can be amplified through the modulation of the drain current in the HEMT. Accordingly, a PVDF-gated HEMT can be used for pressure detection.

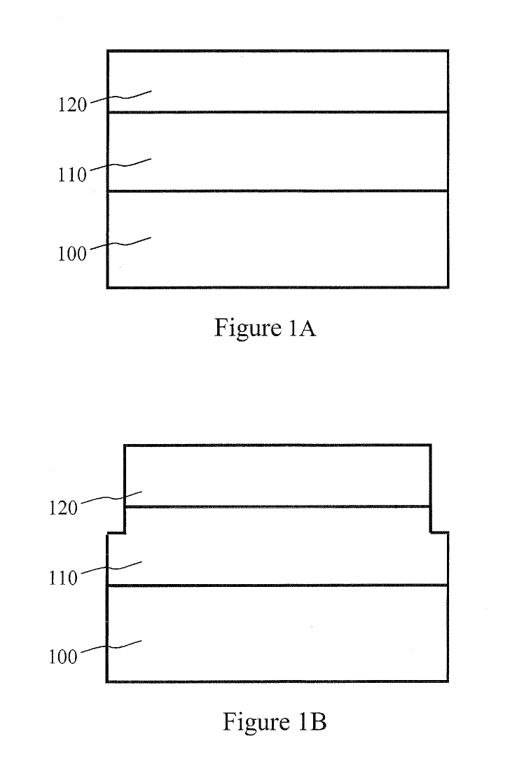

In one embodiment, the HEMT can be an AlGaN/GaN HEMT. Referring to FIG. 1F, the subject pressure sensor can include an AlGaN/GaN high electron mobility transistor structure with PVDF in the gate region. PVDF can be directly cast or dissolved in solvent and then spun on the substrate or distributed on the desired area with liquid plotter or inkjet system. A passivation layer can also be included on the HEMT. The sensing area of AlGaN/GaN HEMT can be in the range of a few hundred microns square to less than one micron square. In many embodiments, the decreased size of the gate area can improve parasitic resistance and can increase detection sensitivity to a certain level.

In operation, a change in fluid pressure on the PVDF film can cause displacement of the PVDF film. The PVDF film can be used to convert this displacement into an electrical signal. The electrical signal from the PVDF can be amplified by the HEMT. Thus the size of the sensitive PVDF pressure sensors can be minimized significantly. The output signal from the HEMT can be wirelessly transmitted to, for example, a display. In one embodiment, a wireless transmitter can be electrically connected to the source or drain of the HEMT.

An AlGaN/GaN HEMT can be fabricated by any method known in the art. According to an embodiment, pressure sensing can be accomplished by functionalizing the AlGaN/GaN HEMT gate region with a piezoelectric thin film. In preferred embodiments, a PVDF thin film is used as the piezoelectric thin film. The PVDF film is highly effective in changing mechanical displacements into electrical signals. The efficiency can be achieved because the g-coefficient for PVDF film is relatively high, about 0.20 VmN⁻¹ when compared with some other piezoelectric materials, e.g. Rochellesalt 0.09, quartz 0.05 and PZT 0.01 VmN⁻¹.

A method for fabricating an AlGaN/GaN HEMT pressure sensor will be described with reference to FIGS. 1A-1F.

Referring to FIG. 1A, an undoped GaN buffer 110 and an undoped AlGaN cap layer 120 can be grown on a silicon substrate 100. In one embodiment, the epi-layers of the GaN buffer 110 and the AlGaN cap layer 120 can be grown by metal-organic chemical vapor deposition (MOCVD) or molecular beam epitaxy (MBE).

Then, referring to FIG. 1B, mesa etching can be performed to isolate device regions. In a specific embodiment, inductively coupled plasma (ICP) etching can be performed for the device mesa isolation using Cl₂/Ar at −90 V DC self-bias, ICP power of 300 W at 2 MHz and a process pressure of 5 mTorr.

Referring to FIG. 1C, source and drain contact pads 130 can be formed on the AlGaN layer 120.

Then, as shown in FIG. 1D, a final metal layer 140 can be formed for providing interconnections.

Referring to FIG. 1E, a photoresist 150 can be coated on the structure and patterned to expose the gate region of the transistor.

Then, referring to FIG. 1F, un-polarized PVDF thin film 160 can be coated at the gate region. In one embodiment, a micro-plotter can be used to deposit the PVDF thin film. In a specific embodiment, the PVDF thin film can be deposited on the gate region with an inkjet plotter. Then, the PVDF thin film can be polarized by, for example, applying an electric field.

In many embodiments, the PVDF film can be cast or spin on the HEMT surface with conventional semiconductor processing technology. These cast or spun films are non-polar and isotropic. Therefore, the piezoelectric nature is induced by application of a high electric field. According to certain embodiments, this can be achieved by a corona discharge (such as taught by Das-Gupta and Doughty in “Piezo- and. Pyro-electric Behavior in Polyvinylidene. Fluoride”, J. Phys. D., Vol. 11, pp. 2415-2525, 1978, which is hereby incorporated by reference in its entirety). According to this method, a wire tip at a distance of about 10 inches from the polymer film serves as a high-voltage electrode. The corona discharge can simply be applied in normal atmosphere and at room temperature. According to this method, direct contact with the surface of the thin film can be avoided and the danger of electric spark to damage the PVDF film and HEMT is reduced.

Although an AlGaN/GaN HEMT is described as the HEMT for use in the aforementioned embodiments, other HEMTs, such as, but not limited to, an AlGaAs/GaAs HEMT, an InGaP/GaAs HEMT or an InAlAs/InGaAs HEMT can be used in place of the AlGaN/GaN HEMT. In addition, although a silicon substrate is described as the substrate 100 upon which the HEMT is disposed, embodiments are not limited thereto. For example, a sapphire or SiC substrate may be used.

In further embodiments, the subject pressure sensing HEMT can be integrated with other sensors in a single chip. The other sensors can include, for example pH, blood glucose detection, oxygen or CO₂ sensors. In one embodiment, the multiple sensors can be fabricated on a single chip by forming a plurality of HEMTs and then individually functionalizing each HEMT for a particular sensing application using any known masking techniques. Other circuitry can also be included, if needed.

In an embodiment, a portable or implantable in vivo blood pressure or internal organ sensor can be realized using a PVDF-gated HEMT. The subject pressure sensor can be implanted in or at a blood vessel to enable continual measurements of the pressure of the blood vessel. According to an embodiment, the subject pressure sensor can be mounted on the head of intravenous therapy or IV therapy.

Embodiments of the present invention can provide a fast response time, portable, low cost, digital signal pressure detector. Further embodiments of the present invention can be used as a wireless based sensor to send the testing results directly to a doctor. For example, the results can be provided to a display.

A greater understanding of the present invention and of its many advantages may be had from the following examples, given by way of illustration. The following examples are illustrative of some of the methods, applications, embodiments and variants of the present invention. They are, of course, not to be considered in any way limitative of the invention. Numerous changes and modifications can be made with respect to the invention.

### Example I

### PVDF-Gated Functionalized HEMT

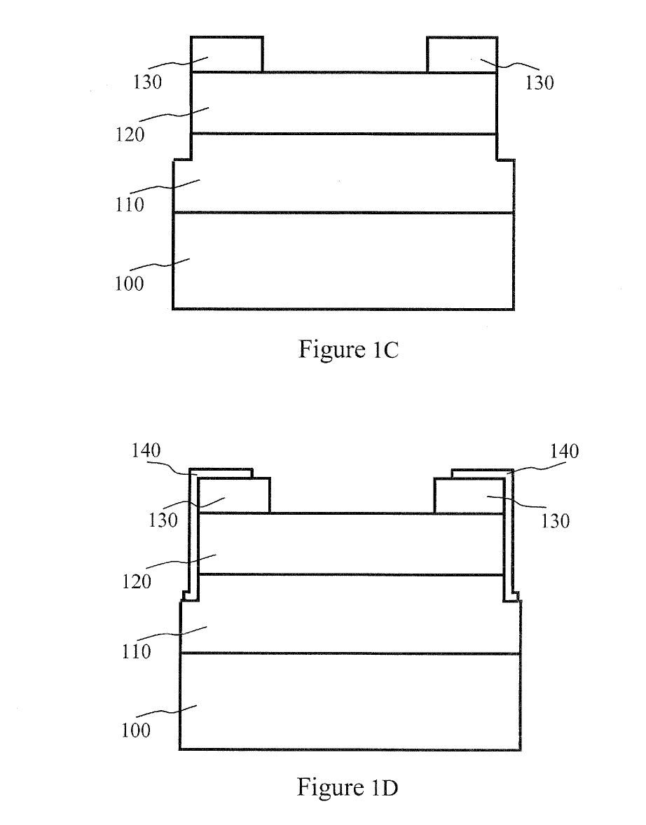

The AlGaN/GaN HEMT structures used for the following examples have a 2 μm thick undoped GaN buffer and a 250 Å thick undoped Al_{0.25}Ga_{0.75}N cap layer. The epi-layers were grown by MOCVD on 100 mm (111) Si substrates. Device mesa isolation was performed using ICP etching with Cl₂/Ar discharges at −90V dc self-bias, ICP power of 300 W at 2 MHz and a process pressure of 5 mTorr. Ohmic contacts each having an area of 50×50 μm² and separated with gaps of 50 μm were formed of e-beam deposited Ti/Al/Pt/Au patterned by lift-off. The contacts were annealed at 850° C. for 45 s under flowing N₂. E-beam deposited Ti/Au was employed as the interconnection metallization for the final metal step. Then, AZ photo-resist was used as the mask to define the gate electrode for PVDF coating. Un-polarized PVDF thin film was coated at the gate region using a micro-plotter. Optical microscope images before and after PVDF deposition on the gate region are shown in FIGS. 2A and 2B. The thickness of PVDF film was about 2 μm.

The PVDF (Sigma-Aldrich) deposited on the gate region had an average molecular weight (MW) of about 534,000 and a glass transition temperature (Tg) of −38° C. A 10 wt % PVDF solution was made by dissolving PVDF powder in n,n-dimethyl acetamide. Although this example used an MW of about 534,000 for the PVDF, different MW of PVDF also can be used. By modifying the MW, the viscosity of the solution can be modified. Then, poly-methyl methacrylate (PMMA) was added in the PVDF solution to improve adhesion of PVDF on the substrate and to increase the proportion of the beta-phase PVDF.

To polarize the PVDF thin film, an electric field was applied to the PVDF film. A wire tip with a diameter of ˜0.2 mm was kept at a distance of 1 cm above the polymer film to serve as a high-voltage electrode. By using a Glassman DC power supply, an electrical field of 0.5 MV/cm was generated to convert the nonpolar alpha form of PDVF into the oriented polar beta form. The polarization time was 30 minutes and the temperature was kept 70° C. to enhance the carbon-carbon rotation.

The effect of exposing GaN/AlGaN HEMTs gated with a polarized PVDF thin film to different pressures using N₂ gas was investigated using the above described devices. The sensitivity, the temporal resolution, and the limit of detection of these sensors for pressure detection were measured.

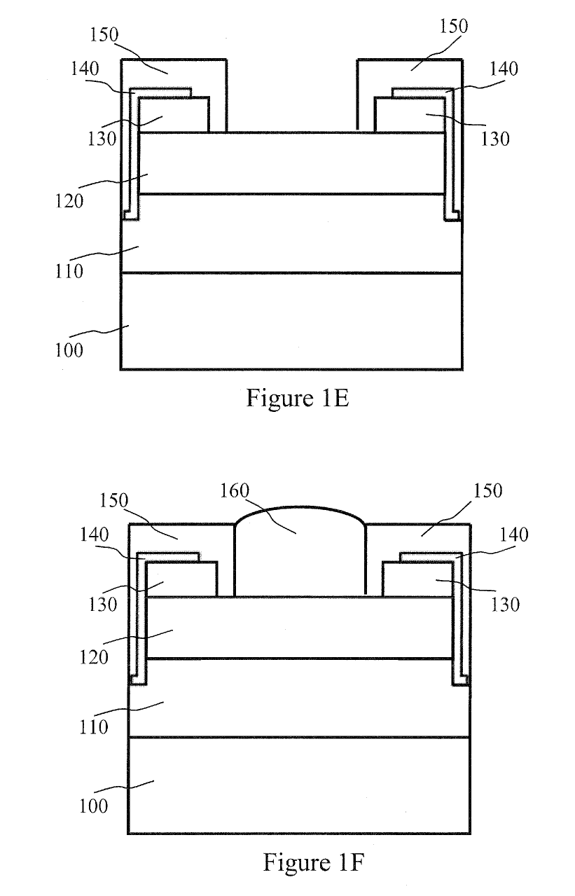

FIG. 3 shows the real time pressure detection using polarized PVDF gated HEMT drain current with a constant drain bias voltage of 500 mV during the exposure to different pressure ambients using N₂ gas. The HEMT sensor was first exposed to 14.7 pound per square inch (psi) under N₂ gas, equivalent to 1 atmosphere (atm), and no change of the drain current was detected for 50 s. This stability was important to exclude possible noise from the mechanical change of the pressure change by N₂. By sharp contrast, there was a rapid increase of HEMT drain current observed in less than 5 s when the ambient pressure was changed to 100 psi. The abrupt current change was due to the piezoelectric contribution of the polarized PVDF. The dipole moment of the PVDF polar bonds increased due to the increase of pressure to compress the polar C—F bonds. The electrons in the two-dimensional electron gas (2DEG) channel of the AlGaN/GaN HEMT are induced by piezoelectric and spontaneous polarization effects. This 2DEG is located at the interface between the GaN layer and AlGaN layer. Positive counter charges at the AlGaN surface layer are induced by the 2DEG. Changes in the ambient of the AlGaN/GaN HEMT affected the surface charge on the device. Thus, the changes in the surface charge due to PVDF piezoelectric effect were amplified, producing a big change in the concentration of the 2DEG in the AlGaN/GaN HEMTs.

As shown in FIG. 3, the sensor was subsequently exposed to different lower pressures at 80 psi, 60 psi, 40 psi and 20 psi, respectively. When the ambient pressure decreased, the drain current correspondently decreased and reached a steady state level within 5 s.

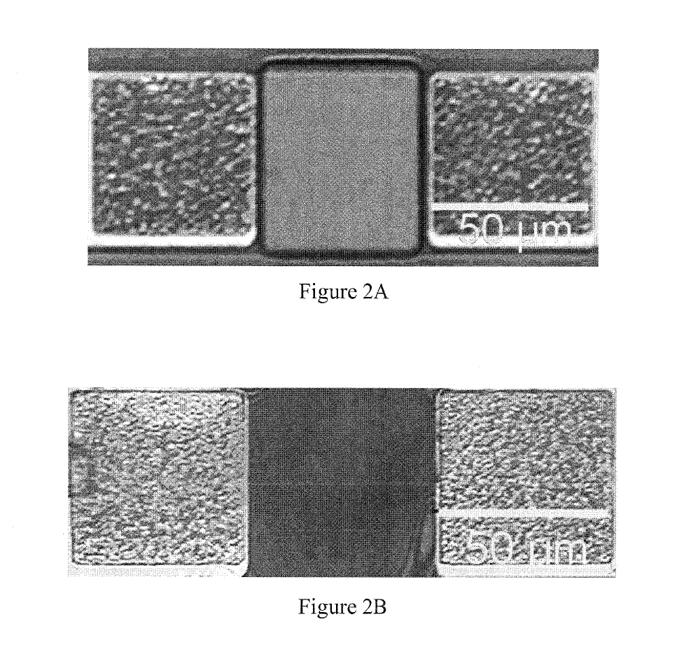

FIG. 4 shows the drain current change of the PVDF gated HEMT sensor as a function of different pressures. In particular, the drain current change (Δμl) is indicated for pressures of 20 psi, 40 psi, 60 psi, 80 psi, and 100 psi. Because the presence of the PVDF gate leads to a logarithmic dependence of current on the pressure, a linear relationship between the drain current change as a function of ambient pressure was obtained.

Advantageously, the AlGaN/GaN HEMTs with a polarized PVDF gate according to an embodiment of the present invention exhibited significant changes in channel conductance upon exposing the device to ambient with different pressures in the range 20-100 psi.

### Example II

### PVDF-Gated Functionalized HEMT—Polarization Effects

Additional pressure sensing measurement tests were conducted where the HEMT sensors in accordance with an embodiment of the present invention were mounted on a carrier and put in a pressure chamber. N₂ gas was used for pressurizing the chamber and a constant drain bias voltage of 500 mV was applied to the drain contact of the sensor.

The sample was mounted on a copper chuck and immersed in a fluorinert electronic liquid F-43 (3M) to prevent arcing during the polarization. A copper wire tip with a diameter of ˜0.2 mm was kept at a distance of 1 cm above the sample to serve as a high-voltage or ground electrode. A Glassman DC power supply was used to apply 10 kV across the sample and the electrode. The fluorinert solution was kept at 70° C. to enhance the carbon-carbon bond rotation in the PVDF during the polarization.

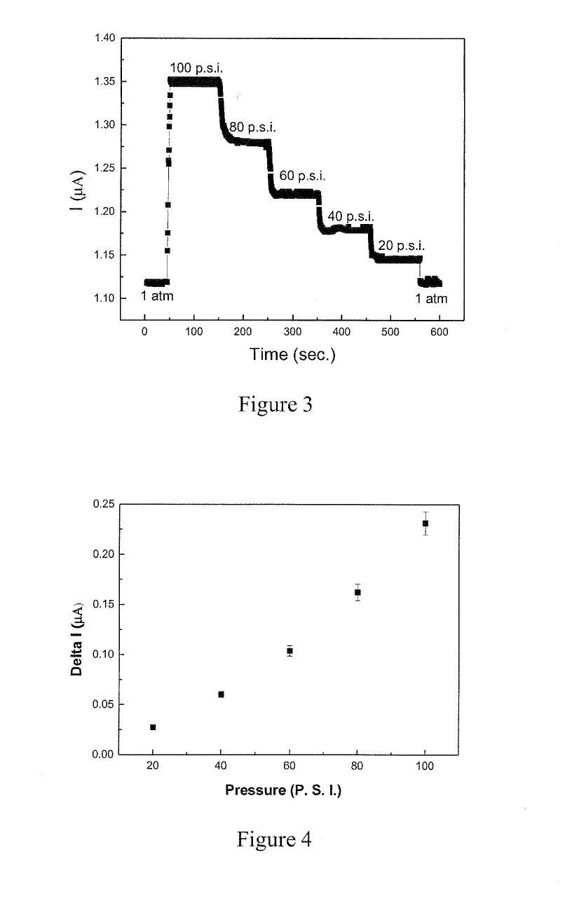

FIG. 5 shows the real time pressure detection with the polarized PVDF gated HEMT. For the results shown in FIG. 5, the PVDF was polarized by grounding the copper chuck holding the sample and applying 10 kV to the copper wire electrode disposed above the sample. As illustrated in FIG. 5, the drain current of the HEMT sensor showed a rapid decrease in less than 5 s when the ambient pressure was changed to 20 psi (gauge). A further decrease of the drain current for the HEMT sensor was observed when the chamber pressure increased to 40 psi (gauge). These abrupt drain current decreases were due to the change of charges in the PVDF film upon a shift of ambient pressure. For comparison, a HEMT sensor without the PDVF coating was loaded in the pressure chamber. There was no observable change of drain current in HEMT sensor without PDVF coating.

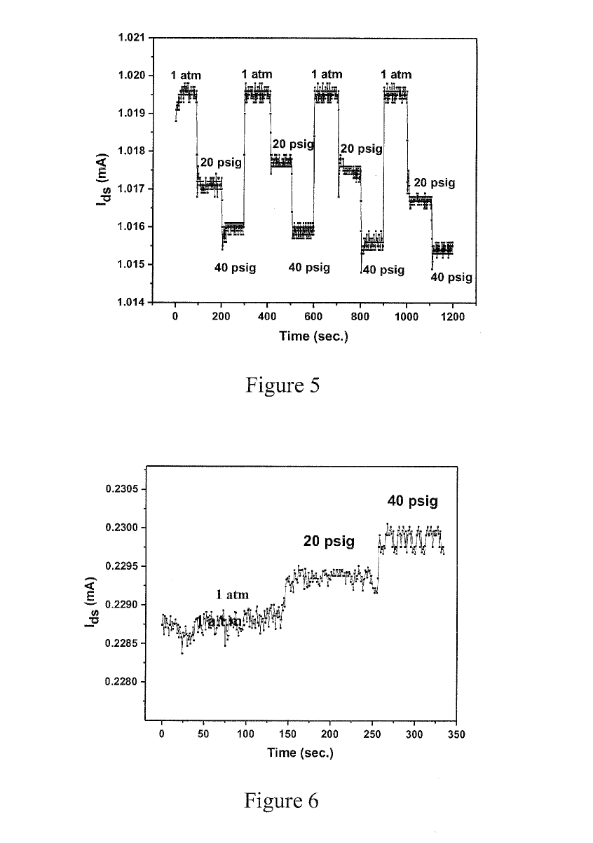

FIG. 6 shows the real time pressure detection with a polarized PVDF gated HEMT having the reverse polarity from the polarized PVDF gated HEMT shown in FIG. 5. As seen in FIG. 6, by reversing the PVDF film polarity, the direction of drain current change was reversed.

Here, the piezoelectric material consists of many small unit dipoles and possesses net positive charges at one end of the materials and negatives at the other end in the direction of the polarization. Under compressive stresses, the length of the piezoelectric sample and the length of the unit dipole in the sample are reduced. Thus, the overall dipole moment per unit volume of the materials reduces accordingly. The change in dipole moment of the materials changes the charges density at the ends of the materials. The sample used in FIG. 5 was polarized by grounding the substrate and applying 10 kV at the top copper wire electrode. Therefore, the PVDF film near the PVDF/AlGaN interface had net positive charges and the other end of the PVDF film possessed net negative charges. Once the PVDF film experienced compressive force, the net charges in the PVDF film were reduced. Thus the total positive charge at the AlGaN surface was decreased and the corresponding 2DEG reduced. By reversing the polarity, such as with the sample used in FIG. 6, the surface of the PVDF film right next to the AlGaN surface had negative charges and the top surface of PVDF film possessed positive charges. When the PVDF film was subject to the pressure, the net negative charges in the PVDF near the PVDF/AlGaN interface were reduced. Thus, the net positive charges at the AlGaN surface increased and drain current of the HEMT increased correspondingly.

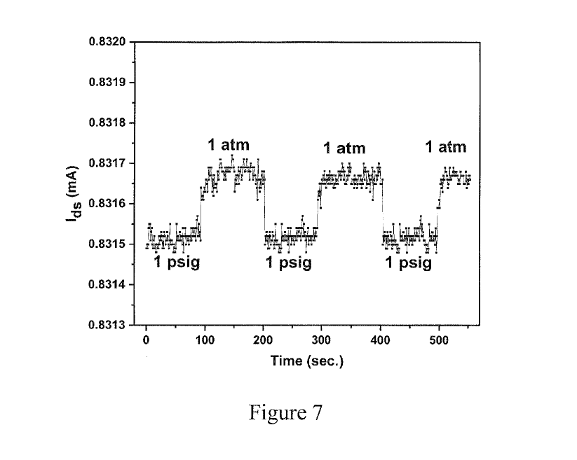

FIG. 7 shows repeatability of the subject pressure sensor. In particular, FIG. 7 shows the drain current response of the PVDF gated HEMT sensor of an embodiment to pressure switching from 1 atm to 1 psi (gauge). The change of drain current for HEMT exposed between 1 atm and 1 psi (gauge) was still considerably larger than the background noise, indicating the ability of the subject HEMT to detect small differences in pressure (e.g., 1 psi (gauge)).

Accordingly, as provided by the foregoing examples, polarized PVDF gated AlGaN/GaN HEMTs showed rapid change in the source-drain current when exposed to different pressure ambient. Accordingly, embodiments of the subject device can be used for bio-sensing applications.

All patents, patent applications, provisional applications, and publications referred to or cited herein are incorporated by reference in their entirety, including all figures and tables, to the extent they are not inconsistent with the explicit teachings of this specification.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

It should be understood that the examples and embodiments described herein are for illustrative purposes only and that various modifications or changes in light thereof will be suggested to persons skilled in the art and are to be included within the spirit and purview of this application.

We claim:

1. A pressure sensor, comprising:
a high electron mobility transistor (HEMT) comprising a piezoelectric biocompatible film on a gate region.

2. The pressure sensor according to claim 1, wherein the gate region has an area in the range of about a few hundred microns square to less than one micron square.

3. The pressure sensor according to claim 1, wherein the gate region has an area of about one hundred microns square.

4. The pressure sensor according to claim 1, wherein the gate region has an area of less than one micron square.

5. The pressure sensor according to claim 1, wherein the piezoelectric thin film comprises polyvinylidene fluoride (PVDF).

6. The pressure sensor according to claim 1, wherein the piezoelectric thin film comprises a metal oxide having piezoelectric properties.

7. The pressure sensor according to claim 1, further comprising:
a wireless transmitter for constant pressure monitoring electrically connected to a source or drain of the HEMT.

8. The pressure sensor according to claim 1, wherein the HEMT comprises an AlGaN/GaN HEMT.

9. The pressure sensor according to claim 1, wherein the HEMT comprises an AlGaAs/GaAs HEMT.

10. The pressure sensor according to claim 1, wherein the HEMT comprises an InGaP/GaAs HEMT.

11. The pressure sensor according to claim 1, wherein the HEMT comprises an InAlAs/InGaAs HEMT.

12. A method of detecting body fluid pressure, comprising:
providing a high electron mobility transistor (HEMT) pressure sensor comprising a piezoelectric biocompatible film in or near a fluid vessel, wherein the piezoelectric biocompatible film changes mechanical displacement caused by fluid in the fluid vessel into electrical signals and the HEMT amplifies the electrical signals.

13. The method according to claim 12, wherein the piezoelectric biocompatible film comprises polyvinylidene fluoride (PVDF).

14. The method according to claim 12, wherein the piezoelectric biocompatible film comprises a metal oxide having piezoelectric properties.

15. The method according to claim 12, wherein the HEMT comprises an AlGaN/GaN HEMT.

16. The method according to claim 12, wherein the HEMT comprises an AlGaAs/GaAs HEMT.

17. The method according to claim 12, wherein the HEMT comprises an InGaP/GaAs HEMT.

18. The method according to claim 12, wherein the HEMT comprises an InAlAs/InGaAs HEMT.

19. The method according to claim 12, further comprising wirelessly transmitting the amplified electrical signals from the HEMT to a display.

20. The method according to claim 12, wherein providing the HEMT in or near a fluid vessel comprises mounting the HEMT on a head of intravenous therapy.
